(19) United States
(12) Patent Application Publication
(10) Pub. No.: US 2007/0077643 A1
(43) Pub. Date: Apr. 5, 2007

(54) Biochemical processing apparatus equipped with display device

(75) Inventors: Yohei Nakamura, Kawasaki-shi (JP); Nobuo Oshimoto, Kawasaki-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/521,392

(22) Filed: Sep. 15, 2006

(30) Foreign Application Priority Data
Oct. 4, 2005 (JP) 2005-291228

(51) Int. Cl. C12M 1/36

(52) U.S. Cl. 435/286.1; 435/286.5; 435/287.2

(57) ABSTRACT

An index region consisting of indexes corresponding to a plurality of processing steps is provided. The job in progress is indicated by changing the display state of the corresponding index. Detailed information on the job in progress is displayed in an information display region, which is provided in a different region from the index region. The display state of the index corresponding to the step in progress is changed with time in accordance with the proceeding of the processing step, thereby indicating the progress of the treatment. When the user selects the index corresponding to a job, the information on the job is displayed in the information display region.

### BACKGROUND OF THE INVENTION

1. Field of the Invention

The present invention relates to an apparatus having a plurality of processing steps, and particularly, a biochemical processing apparatus using a DNA chip.

2. Description of the Related Art

When the base sequence of genetic DNA is analyzed or gene diagnosis is performed simultaneously with respect to multiple items, DNA having a desired base sequence must be detected by use of a plurality of types of probes.

As a means for providing a plurality of type of probes used in the testing item, a DNA chip has received attention (for example, Japanese Patent Application Laid-Open No. 2001-017166).

Also, in high-throughput screening of medicaments or the likes or combinatorial chemistry, solutions of a number of target proteins or medicaments are arranged and must be screened orderly.

Actually, 96 types, 384 types and 1,536 types of medicaments are used.

In order to perform the orderly screening, a method of arranging a plurality of types of medicaments in an array, an automatic screening technique and apparatus thereof the array, and software for controlling a series of testing items and statistically processing the results have been developed.

Parallel screening as mentioned above is basically performed by subjecting a substance to be evaluated to a so-called probe array in which known probes (which serves as a screening means) are arranged in the form of array.

Whether the substance (to be evaluated) reacted (acted) with a probe(s) or not can be detected under the same conditions by use of the probe array.

Generally, which probe is used, that is, which reaction (action) is used, has been previously determined.

Therefore, probes to be arranged on a single probe array belong to a single type of substances such as a group of DNA probes different in base sequence, when they are roughly categorized.

As the substance to be employed as the single group of probes, for example, DNA, protein, synthesized chemical substance (medicament) may be mentioned. In most cases, a probe array consisting of a group of a plurality of types of probes is used.

However, depending upon the testing item, a single DNA having the single base sequence, a single protein having the single amino acid sequence, or a single chemical substance may be arranged in many locations to form an array which will be then used.

These are principally used in screening medicaments.

As a probe array formed of a group of a plurality of types of probes, for example, a group of DNA fragments having different base sequences, a group of proteins having different amino acid sequences, and a group of different chemical substances may be used.

A plurality of types of substances are usually arranged in an array form on a substrate in accordance with predetermined order.

Of the arrays, a DNA probe array is used in analyzing the base sequence of genetic DNA or in making gene diagnosis simultaneously with respect to multiple items with high reliability.

In a gene analysis method using a probe, first a high molecular substance to be used as a target is extracted from a specimen and amplified as needed. Then, to the high-molecular target substance, a detectable label such as a fluorescent label is attached.

The high molecular target substance is hybridized with a probe to bind them. The reaction of the high molecular target substance with the probe can be determined by detecting the label.

When DNA probes having specific base sequences of a plurality types of bacteria and viruses are used, the bacteria and viruses contained in the specimen can be determined and therefore therapeutically useful.

A step of extracting DNA from a human differs depending upon the type of target DNA. For example, when blood is used as a specimen, blood cells are lysed and, if necessary, crushed.

When the presence or absence of bacteria is checked, blood cells are cultured to amplify them.

Recently, DNA is isolated by magnetic separation. In this method, after the cells are lysed, DNA is allowed to adsorb to magnetic particles coated with silica. While the magnetic particles are collected by a magnet, extra components are removed.

Most of DNA extracted from a human specimen is accounted for by the human genome. It is therefore difficult to detect a DNA possibly present in a minute amount, such as bacterial DNA, directly from the DNA extract.

To overcome the difficulty, an extracted DNA is generally amplified by the polymerase chain reaction (PCR) method (Science Vol. 230, p 1350-1354 (1985)).

This is a method of amplifying a desired DNA to be amplified by powers of 2 by mixing primers complementary to the DNA and a relevant enzyme with a specimen, and then subjecting to a heat cycle.

After completion of the amplification step, a purification step is performed in the same manner as in the extraction step as mentioned above. Thereafter, a primer tagged with a detectable fluorescence label is added, and subjected again to a heat cycle to obtain a DNA fragment tagged with the fluorescence label.

The DNA fragment is then subjected to a hybridization step to bind to a probe. The hybridization is performed in a solid-phase reaction in accordance with a DNA chip method as mentioned above.

The reaction is performed by mixing DNA tagged with a fluorescent label with a reaction reagent, and introducing the mixture onto the DNA chip.

After completion of the hybridization reaction, excitation light is applied to the DNA chip and fluorescence emitted from the DNA chip is detected. Is this step, which probe of the probe array on the DNA chip emits fluorescence is checked. In this manner, the presence or absence of DNA and the quantity of DNA can be determined.

Various methods have been developed for performing these steps automatically and not put into practical use in medical institutions and research institutions.

However, operations to be performed between steps, such as mixing of reagents and setting of reagents to an apparatus, are still manually performed. When a large number of specimens are tested, extraordinarily large inputs of time and labor are required at present.

Recently, a full automatic DNA testing apparatus capable of automatically carrying out these steps has been developed.

The full automatic DNA testing apparatus is desired to attain reduction of testing time and usability. The reduction of testing time is mostly dependent on a process. Various new methods have been proposed to reduce testing time.

On the other hand, to improve usability, handling and discarding method of reagents and specimens have been conceived from safety and convenience point of views. As another aspect to improve usability, information displayed on a display device may be mentioned.

In a testing apparatus in which a plurality of specimens are processed in parallel by sequentially processing them through the aforementioned 4 steps, it is important for the user to know which specimen is treated in which step at a glance.

However, it is not preferable to enlarge a display device or demand the user to perform a complicated operation.

As a display system, an interactive-basis display system has been proposed (Japanese Patent Application Laid-Open No. H11-232366) in which items setup in an electronic form are displayed in an interactive manner. On the other hand, a display method is disclosed in Japanese Patent Application Laid-Open No. 2003-050242, wherein information on a specimen of concern, more specifically, including a step where the specimen is now processed can be easily checked.

Various testing items can be carried out by the full automatic DNA testing apparatus by providing such materials as reagents and a DNA chip suitable for each DNA to be tested.

Therefore, the types of reagent and DNA chip must be changed depending upon the DNA to be tested and a protocol must be changed accordingly.

If an apparatus is provided with all types of reagents and DNA chips preset therein, the apparatus will be inevitably enlarged. To realize various test items by a small apparatus, reagents and a DNA chip suitable for each testing item must be set by the user.

On the other hand, to reduce the testing time, an apparatus must be developed in which a plurality of specimens can be processed in parallel by setting a next specimen when a first specimen has processed to a predetermined step.

One or more specimens may be used in a single specimen treatment. The treatment of one or more specimens will be called a “job”, hereinafter.

When such parallel jobs are performed for a plurality of specimens, a long time is required. Therefore, during the long-time screening step, it is necessary to clearly display at a point of which time a reagent and a DNA chip for the next specimen are injected and set.

More specifically, it is desired that information on whether the job for testing a next specimen can be started and instructions for injecting or setting and discharging or disposing (discarding) of a reagent and a DNA chip are displayed on the display device.

In addition, it is desired that the step in progress can be checked at a glance, and that the estimated necessary time and information on a specimen already treated can be checked on the display device.

List-up information alone is not sufficient. To display the testing jobs of specimens sequentially set into the apparatus simultaneously in a single display, a large screen is required.

In the above method for interactively displaying items set in an electronic form, the user must set items one after another for a plurality of required steps. Therefore, the interactive display is not suitable for an automatic apparatus.

Furthermore, the method that enables information on the processing step which the specimen is undergoing to be easily checked requires a large display device. As a result, enlargement of the entire apparatus still remains as a problem to be overcome.

### SUMMARY OF THE INVENTION

An object of the present invention is to provide a biochemical processing apparatus equipped with a display device on which operation state of the apparatus can be easily checked.

According to an aspect of the present invention, there is provided a biochemical processing apparatus executing a plurality of processing steps, comprising a display device; and a display circuit for displaying an index corresponding to each of the processing steps and information regarding the processing step separately in different regions of the display device, wherein the display circuit changes the display state of the index depending upon the progress of the processing step(s) and displays the information regarding the processing step corresponding to the index selected on the display device.

The information regarding the processing step preferably includes a warning and a message to the user. The display circuit preferably shows information of a step of a previous job in the information display region when a plurality of steps are executed simultaneously. When a warning and a massage are displayed, the user's attention is preferably drawn by displaying the corresponding index thereof so as to give a warning.

The index is preferably a tab.

The display device preferably possesses a touch panel function. The index is preferably selected by pressing the touch panel.

The index is preferably selected by operation of a mouse.

The index is preferably selected by operation of a specialized controller.

The index is preferably selected by pressing push buttons arranged around the display device.

The information regarding the processing step preferably includes the estimated time necessary for step in progress.

The information regarding the processing step preferably includes the estimated time necessary for the whole steps.

The information regarding the processing step preferably includes the time at which a reagent is injected and a DNA chip are set.

The information regarding the processing step preferably includes the number of specimens to be treated. The information regarding the processing step preferably includes the identification numbers of specimen treatment.

A step presently unexecuted, a selected step in progress, and a step in progress not selected are preferably indicated by the index.

The display circuit preferably displays a system message of the entire apparatus in the region different from a region of the indexes and the information display region for displaying information regarding the processing step.

Further features of the present invention will become apparent from the following description of exemplary embodiments (with reference to the attached drawings).

### BRIEF DESCRIPTION OF THE DRAWINGS

FIG. 1 is a schematic illustration of a biochemical processing apparatus according to the present invention.

FIG. 2 is an illustration of a display format according to the present invention.

FIG. 3 is an illustration of a display screen for setting the number of specimens.

FIG. 4 is an illustration of a display screen for injecting a reagent.

FIG. 5 is an illustration of a display screen for an extraction step.

FIG. 6 is an illustration of a display screen for an amplification step.

FIG. 7 is an illustration of a display screen for a hybridization step.

FIG. 8 is an illustration of a display screen during a detection step.

FIG. 9 is an illustration of a display screen for instructing to discard a cassette.

FIG. 10 is an illustration of a display screen for setting the number of specimens when a plurality of jobs are executed.

FIG. 11 is an illustration of a display screen for an extraction step when a plurality of jobs are executed.

FIG. 12 is an illustration of a display screen displaying a message “INJECTING REAGENT” when a plurality of jobs are executed.

FIG. 13 is an illustration of a display screen for a detection step when a plurality of jobs are executed.

FIG. 14 is an illustration of a display screen for instructing to discard a cassette when a plurality of jobs are executed.

FIGS. 15A, 15B and 15C are illustrations explaining types of indexes.

FIG. 16 is a block diagram showing the constitution of the display system.

### DESCRIPTION OF THE EMBODIMENTS

### EXAMPLE 1

A biochemical processing apparatus according to the present invention will be schematically explained by way of drawings.

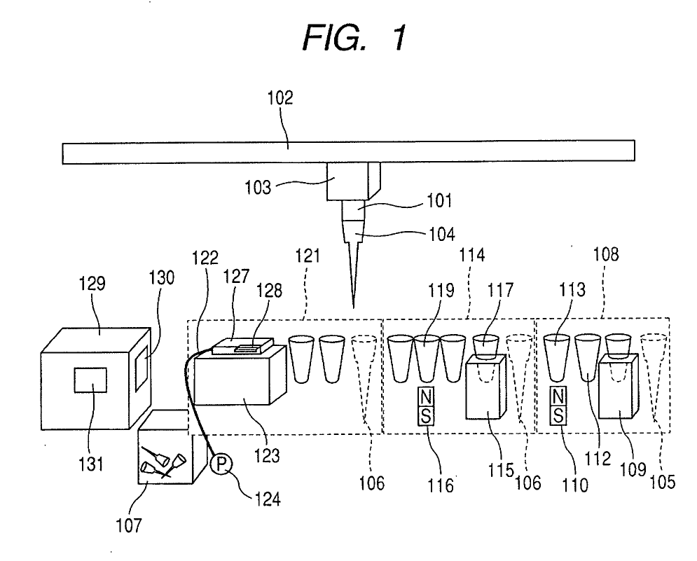

FIG. 1 shows a schematic illustration of a biochemical processing apparatus according to the present invention.

The biochemical processing apparatus according to the present invention comprises treatment units, which are configured so as to correspond to individual processing steps, for example, by additionally attaching a magnet, heating block and pump, etc., and a pipette unit for transferring a reagent from processing step to processing step and handling a liquid, more specifically, sucking, ejecting and stirring of a solution.

The figure illustrates schematically the apparatus to treat a single specimen for brevity. However, the same configuration is basically used even for an apparatus where a plurality of specimens (for example, 6 specimens) are simultaneously treated only by adding syringes and wells.

The pipette unit 101 has a syringe for sucking or ejecting a solution, a mechanism of opening the cover of a reagent package, and a mechanism of ejecting a disposable pipette tip.

The pipette unit 101 must be moved in the X-axis direction and the Z-axis direction to move between the individual treatment units for sucking and ejecting a solution and opening the cover. Therefore, the pipette unit 101 is immobilized onto a Z-stage 103 formed on an X-stage 102.

In the case where the treatment units are arranged within an X-Y plane, the pipette unit 101 is mounted on the X-Y stage. By virtue of the configuration of the apparatus, the pipette unit can be moved up/down and right/left between the treatment units in the apparatus to transfer a reagent.

A pipette tip 104 (which is supplied together with a reagent) is picked up from a pipette tip storage 105 and fit to the pipette unit 101 by pressure. A new pipette tip 104 is used for each specimen.

When all steps are completed, the pipette tip is discarded in a dump 107 by the ejection mechanism provided in the pipette unit 101.

Since the pipette tip 104 for handling a reagent and reagents used in the steps are disposable, contamination rarely takes place.

Since a pipette tip 104 is replaced with a new one when a new specimen is used, when a plurality of specimens are treated in parallel, the pipette tips 104 for use in the specimens must be replaced with new ones every time a new job (processing step) is started.

An example of such replacements is in a case where first job is hybridization and a second job is purification of a 1st PCR amplified product.

In this case, pipette tip 104 used in the first job is tentatively placed on a temporary tip-laydown site 106 and a pipette tip 106 for the second job must be fitted.

The figure shows a single pipette tip. However, the number of pipette tips is not limited to one and a plurality of pipette tips may be used.

When a pipette tip is discarded and a new pipette tip is used for new reagent, an operation for replacing the pipette tip 104 using the temporary pipette tip-laydown site 106 is not required.

In an extraction unit 108, a heating block 109 for lysing cells of a specimen and a magnet 110 for performing extraction using magnetic particles coated with silica are provided. A lysis reagent and the specimen are mixed and the mixture is heated on the heating block 109. In this manner, the cellular membrane of the specimen is lysed.

To explain more specifically, first, the specimen is sucked by the pipette unit 101 and transferred into a well previously filled with the reagent by the pipette unit 101 to mix them, and then, the mixture of the specimen and the reagent is repeatedly sucked and ejected by use of a pipette syringe (pipette unit), thereby stirring the mixture.

After lysis treatment, the resultant mixture is blended with the magnetic particles coated with silica in a magnetic purification well 113 to adsorb DNA, and then, the magnet 110 is allowed to approach near the well 113 by magnet driving means, thereby capturing the magnetic particles.

Remaining solution is sucked and discarded. In this manner only DNA adsorbed onto the magnetic particles can be extracted.

Thereafter, the aforementioned purification step is repeated using a washing solution to remove extra substances except DNA. Finally, DNA is eluted from the magnetic particles with an eluent. The resultant solution is sucked in the state where the magnetic particles are attracted by the magnet 110 to obtain the extracted DNA. In this manner, the extraction step is terminated.

The figure shows a single reagent well 112 for brevity. However, the reagent wells are prepared as many as they are required for a purification solution and eluent, etc.

In an amplification unit 114, a heating block 115 for performing PCR and a magnet 116 for use in purification with magnetic particles coated with silica are provided.

Amplification reagents including an enzyme, primers and the like are mixed with the extracted product (obtained in the previous step) in an amplification well 117 on the heating block 115.

Subsequently, the mixture is subjected to thermal cycling (where the temperature is changed between 94° C. to 64° C.) on the heating block 115. In this manner, a 1st PCR amplification is performed.

During the 1st PCR amplification, the amplification well 117 is covered with a lid (not shown in the figure) for preventing vaporization and lid heating means (not shown in the figure) for preventing liquid droplets from attaching onto the lid is used.

The DNA extracted from a human is accounted for mostly by the human genome. Therefore, even if it is desired to detect a bacterial species of an infectious disease, the target bacterial DNA is rarely contained.

Therefore, the target bacterial DNA must be amplified to a detectable level by repeating the thermal cycle of the 1st PCR amplification, for example, 40 times.

After the 1st PCR amplification, a purification step is performed to remove the amplification reagent etc. not participated in the reaction.

The purification step is performed in the same manner as in the purification step performed in the extraction step. To describe more specifically, purification is performed by permitting the magnet 116 to approach near a purification well 119 containing the magnetic particles coated with silica and discarding the solution except the magnetic particles captured by the magnet.

The DNA thus purified is eluted from the magnetic particles coated with silica in the same manner as in the extraction step and then subjected to a 2nd PCR step.

In the 2nd PCR step, the same amplification reagents as the 1st PCR step are used except the primer. The primer used herein is complementary to a probe immobilized onto a DNA chip and tagged with a detectable fluorescent label.

Although a single amplification well 117 is shown in the figure, the amplification well 117 as used in the 1st PCR is not used. A new amplification well 117 is used for performing thermal cycle treatment.

The 2nd PCR is performed principally to increase a single stranded DNA required for detection. The PCR is performed by repeating the thermal cycle, for example, 25 times, followed by attaching a fluorescent label to the amplified DNA.

In a hybridization unit 121, a cassette 127 in which a DNA chip 128 is installed, a heating block 122 for use in binding DNA with the DNA chip, and a pump 124 for use in dehydration are provided.

The cassette 127 is provided with an opening and a channel for delivering a hybridization solution containing a target DNA onto the DNA chip 128 and also a reaction chamber for performing a hybridization reaction on the DNA chip 128 with the hybridization solution held thereon.

The cassette 127 has an opening for discarding the hybridization solution after completion of the reaction and pump means 124 connected thereto which is used to introduce or discard by suction the solution and for dehydration.

The solution to be introduced into the cassette 127 is not limited to the hybridization solution, but includes a washing solution and alcohol for dehydration.

First, a fluorescent-labeled product amplified in the previous step and the hybridization solution are mixed and stirred and then supplied to the cassette 127.

To introduce the hybridization solution into the reaction chamber of the DNA chip 128 in the cassette 127, the pump 124 is driven. The hybridization solution is introduced by use of the negative pressure thus generated.

The negative pressure is not necessarily large enough to suck up the liquid but enough to allow the liquid to remain in the chamber. Therefore, not a vacuum pump (controlled by pressure) but a syringe pump (controlled by the suction amount) is desirably used as the pump 124.

After the hybridization solution is introduced, a heating block 123 is turned on to control the temperature suitable for the reaction, for example, 50° C. The hybridization reaction is performed while accelerating the reaction by moving the solution by the pump 124.

After completion of the hybridization reaction, the washing solution and alcohol for dehydration are introduced into the chamber to wash and dry the DNA chip 128, and then, completely sucked by use of the negative pressure of the pump 124, without fail.

Liquid droplets, if they remain on the DNA chip 128, inhibit detection and may prevent obtaining right results. The negative pressure herein is generated in order to suck the solutions, so that a vacuum pump (controlled by pressure) is desirably used.

In the detection unit 129, an optical system such as laser and a lens for use in detection of fluorescence, and a main scanning unit and a sub-scanning unit for scanning the DNA chip are provided.

After completion of the treatment in the previous step, first, an inlet 130 of the detection unit 129 is opened and then, the cassette 127 is transferred into the unit 129 by transfer means.

After completion of the transfer, the inlet 130 is closed. The detection area where the DNA chip 128 is located in the cassette 127 is scanned by the main scanning unit while the cassette 127 is fed by the sub-scanning unit. After completion of the detection, an outlet 131 is opened and the cassette 127 is taken out. In this manner, the entire steps are completed.

In every treatment unit, a plurality of specimens can be processed in parallel. Whether each specimen can be processed or not only depends on whether the pipette unit 101 used in common is available or not. It is because PCR amplification includes a long-time of heat cycling, during which the pipette unit 101 is not used.

Furthermore, most of the time required by hybridization is accounted for by a heating step with the heating block 123. The pipette unit 101 is not used during the heating. Neither is the pipette unit 101 used in the detection step.

Taking advantage of such open time, a plurality of jobs can be carried out in parallel.

For example, when hybridization is started as a first job, extraction is performed as a second job during the hybridization. When the first job proceeds to detection, the second job is allowed to enter amplification.

By performing possible jobs one after another as mentioned above, time is saved, with the result that the overall time required for screening of a plurality of specimens can be shortened.

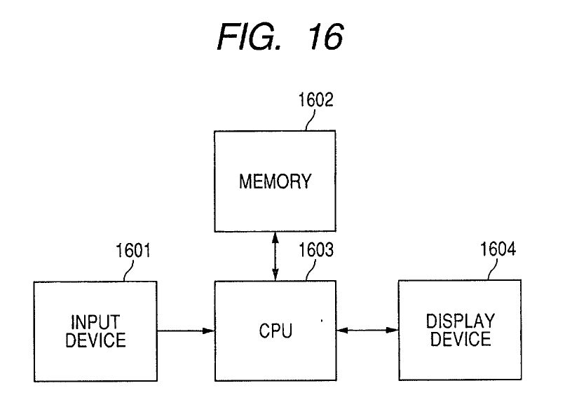

FIG. 16 is a block diagram showing the constitution of the display system of the apparatus in which a plurality of jobs including the aforementioned processing steps can be simultaneously performed. The display system is constituted of an input device 1601, a memory device 1602, a CPU 1603 and a display device 1604.

The input device 1601 outputs information showing the operation state of each of the aforementioned devices to the CPU 1603. The memory device 1602 stores the operation program of the CPU 1603 and temporarily stores a program required for display operation.

The CPU 1603 controls the display of the display device 1604 in accordance with input data from the input device 1601 and the display device 1604 based on the programs stored in the memory device 1602.

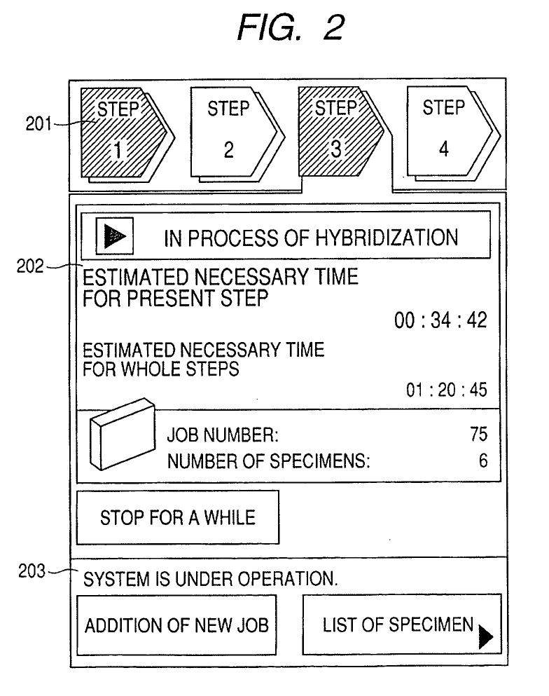

The display of the display device 1604 is controlled by the CPU 1603. The display method of the present invention will be explained with reference to FIG. 2 showing a display screen of the display device 1604.

The liquid crystal panel of the display device 1604 is arranged, for example, in the front or over the top of the device at a position where the user can easily handle the panel. Note that the display device 1604 used herein is supposed to have a touch panel interface, which can be operated by pressing buttons by a finger or a specific pen.

By virtue of the touch panel interface, a direct relation between display and operation is provided, so that the user can easily understood how to operate it.

Since operation can be performed by directly pressing the touch panel or touching an index thereon, a directly-sensible operational environment can be provided.

However, the screen operation is not limited to the one via the touch panel. A mouse or a specific controller connected to the device may be used. Alternatively, use may be made of push buttons, which are (previously) arranged around the display device 1604 so as to correspond to the display screen.

When a mouse is used, it may be handled as if it were with a PC unit.

Use of a specific controller can provide the device with a good operational environment which is dedicated to the device.

When the push buttons is used, since buttons are discretely arranged, miss-touch of the buttons can be reduced and right operation of buttons can be secured.

Indexes are used herein. As the indexes, tabs are used. Tabs 201 (STEPS 1 to 4), which indicate individual steps performed by the apparatus, are arranged on the top of the display screen. By virtue of the tab index, display can be more easily understood.

To explain more specifically, STEP 1 indicates an extraction step, STEP 2 an amplification step, STEP 3 a hybridization step, and STEP 4 a detection step. The name of the step or icon indicating the details of the step may be used as the caption of a tab.

In the figure, the display indicates that STEP 1 (extraction step) and STEP 3 (hybridization step) are in progress. It is apparently shown that two jobs are simultaneously performed.

An information display region 202 is shown below the tab region of STEP 3 (in progress). Both regions appear to be displayed continuously in the same display plane. In the information display region 202, not only the details of the step (in progress) but also the time necessary for terminating the present step and the time necessary for terminating the whole steps are displayed. Furthermore, the identification number of the job (specimen processing) in progress and the total number of specimens undergoing processing are also displayed in the information display region 202.

Since the identification number of a job (specimen) is displayed, which specimen is subjected to the job in progress can be confirmed.

Since the total number of specimens to be processed is displayed, the number of specimens subjected to the job in progress can be checked and a necessary number of reagents can be supplied when injecting the reagent.

Since the time required until the step in progress is completed is displayed, the user can obtain the information on how much time is left until the specimen is transferred to the next processing.

Since the time required until all steps are completed is displayed, the user can capture the information on what time the all steps are completed.

The number of specimens in progress represents the number of specimens to be subjected to the job in progress in a biochemical treatment apparatus, which is assumed to treat at most 6 specimens per job.

The tab 201 has three display styles. In the first style, as exemplified by STEP 3 tab, a tab region is displayed in the same continuous display plane with the information display region, indicating that STEP 3 is in progress and the details of the job in progress for the step of this tub are displayed in the information display region. The first style is called as an “active tab”.

The second style, as exemplified by STEP 1 tab, a tab region is not displayed in a contiguous display plane with the information display region 202, indicating that STEP 1 is in progress but the details of the job in progress for the step of this tub are not displayed in the information display region. The second style is called as a “non-active tab”.

The third style is the cases of STEP 2 tab and STEP 4 tab, indicating that no job is in progress.

As described above, by virtue of three display styles: a step which is not executed at present; a step in progress and selected to display detailed job information in the information display region: and a step in progress but not selected to display the detailed job information in the information display region, the user can visually capture the information on which jobs are in progress, and know detailed information on a specific job by selecting it from a plurality of jobs in progress.

Below the information display region 202, there are a system message region 203 and a button indicating “LIST OF SPECIMEN”. In the system message region 203, the massage on the driving state of the system is displayed. When the “LIST OF SPECIMEN” button is pressed, a list of specimens screened by the apparatus in the past is displayed.

Since the system message region 203 (for displaying the massage on the entire system of the screening apparatus) is arranged separately from the index region and the information display region, whether the driving state of the system is normal or not can be displayed.

In the system message region 203, a message to the user, including a warning and an instruction, is displayed. In this way, a warning from the system, or an indication about the state of the system waiting for completion of the processing can be given to the user.

In the figure, there is a button for “ADDITION OF NEW JOB”, which is presently inactive but becomes active when the conditions as described above for starting the next job are satisfied.

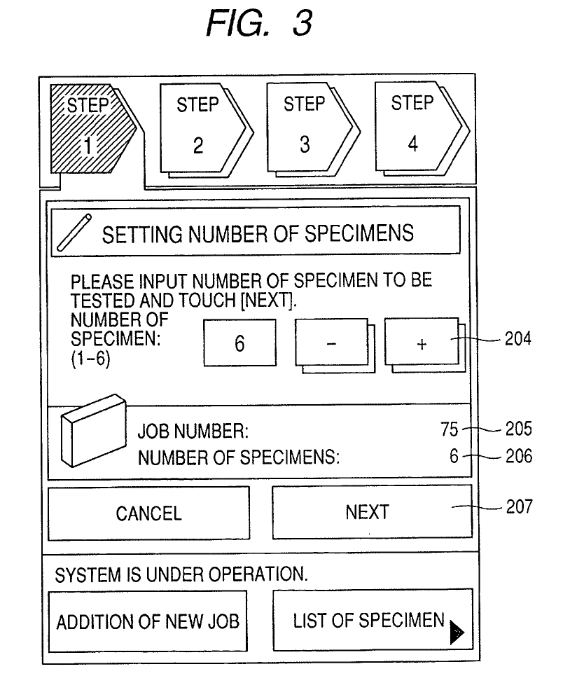

As the treatment proceeds to the later steps, the display state changes as shown in FIGS. 3 to 9.

When the user selects a start of screening on the initial screen of a display device, a first setup display screen as shown in FIG. 3 appears. Since this is the first display screen, the tub of STEP 1 showing the extraction step is active.

This time, a biochemical processing apparatus is assumed to treat at most 6 specimens per job. Under the assumption, the number of specimens to be set into the job to be started is set.

The number of specimens is set by pressing a button 204 (consisting of minus and plus keys) in the information display region. When the number of specimens is set herein, this number also appears in the column of “the number of specimens” 206 (displayed together with “the job number” 205) in the lower portion of the information display region (displaying the detailed information on the job).

After completion of number setting, a button 207 “NEXT” is pressed in accordance with the message. Then, the next display screen for injecting a reagent appears.

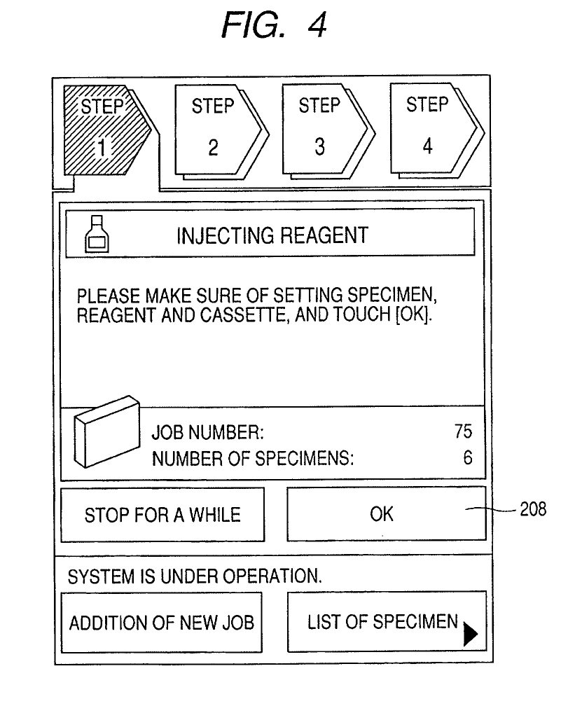

The display screen for injecting a reagent is shown in FIG. 4. The number of specimens set in the previous display screen are set into the apparatus and the corresponding number of reagents are injected into the apparatus. After completion of the setting and injecting, an “OK” button 208 is pressed to initiate the treatment.

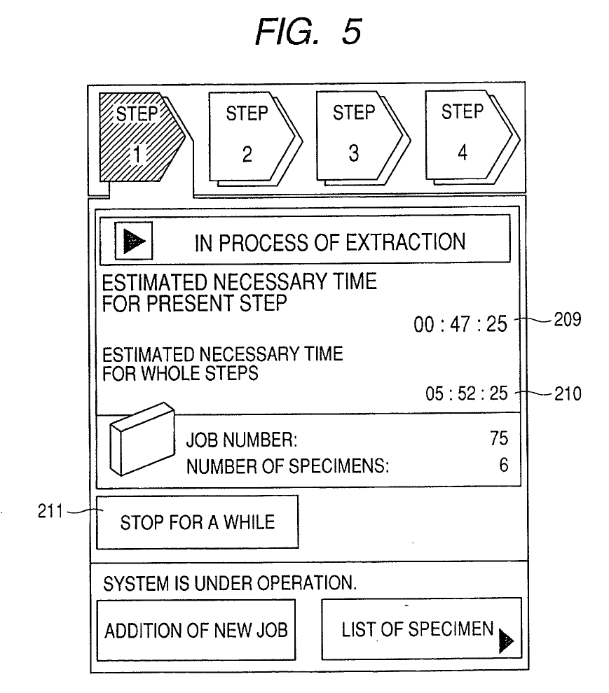

The display screen for an extraction step is shown in FIG. 5. In the information display region, the estimated necessary time 209 of the extraction step and the estimated necessary time 210 of the whole steps are displayed and counted down. When an emergency situation takes place, the user can press the “STOP FOR A WHILE” button 211 to interrupt the treatment. If no problem is confirmed, the treatment may be restarted. If a problem is found, the step is completely terminated.

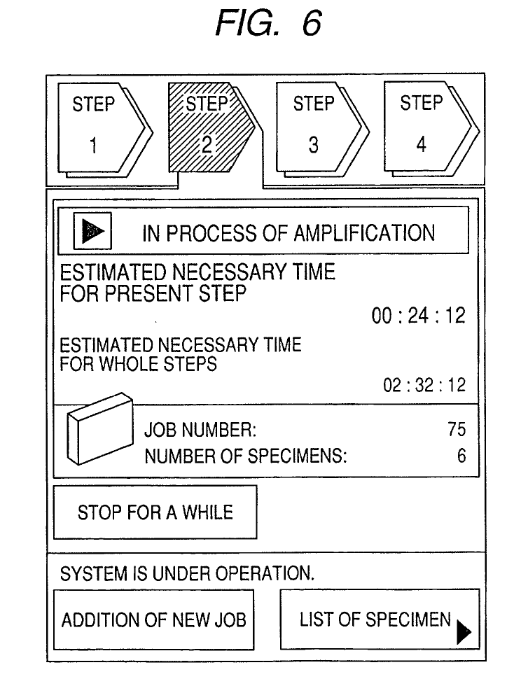

After the extraction step, the operation proceeds to an amplification step. The display screen of the amplification step is shown in FIG. 6. A message “IN PROCESS OF AMPLIFICATION” is displayed in the information display region; at the same time, the STEP 1 tab automatically becomes active in place of the STEP 2 tab.

By this mechanism, the user can visually recognize which job's massage is displayed on the screen.

The STEP 1 tab is deactivated. Instead, the STEP 2 tab is activated. The estimated necessary time of the amplification step is displayed and counted down.

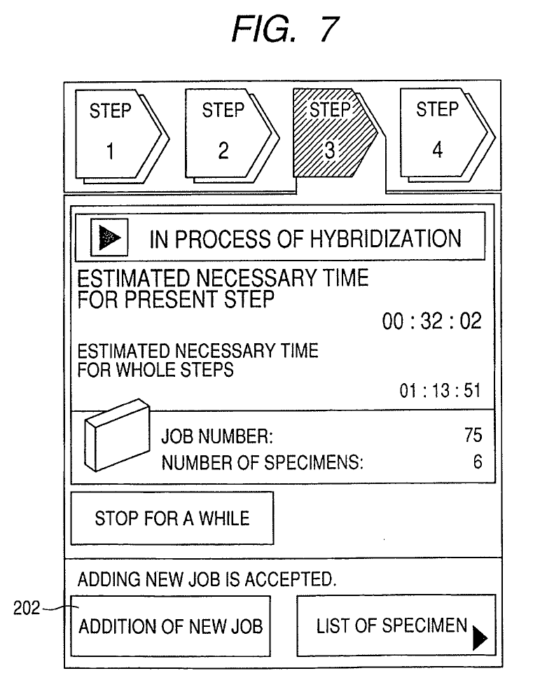

After the amplification step, the operation proceeds to a hybridization step. The display screen of the hybridization step is shown in FIG. 7.

In the same manner as described when the extraction step proceeds to the amplification step, a message “IN PROCESS OF HYBRIDIZATION” is displayed in the information display region; at the same time, the STEP 2 tab automatically becomes active in place of the STEP 3 tab.

The STEP 2 tab is deactivated. Instead, the STEP 3 tab is activated. In the hybridization step, since the extraction step and amplification step are completed herein, the pipette unit is not used.

Since the pipette unit is not used, a next job can be started at this stage. The message “new job can be added” is displayed in the system message region, which is arranged in the lower portion of the display screen. A “ADDITION OF NEW JOB” button 212, which has been inactive (not allowed to press), is activated (allowed to press).

When the user has another a specimen to be screened, he/she presses the “ADDITION OF NEW JOB” button 212 and sets the specimen into the apparatus.

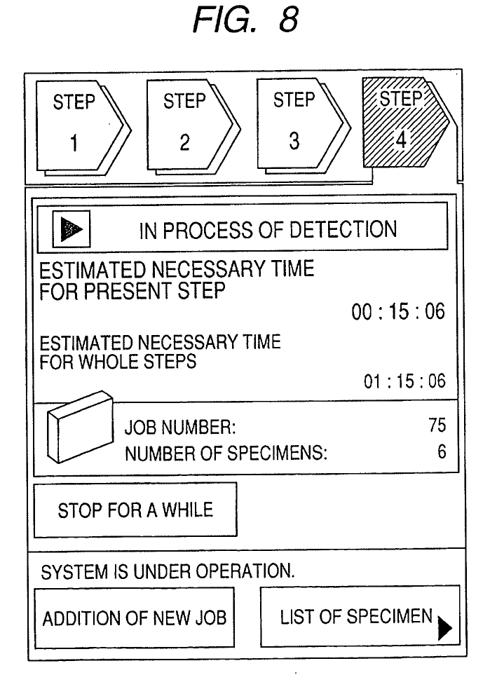

After the hybridization step, the operation proceeds to the detection step. The display screen of the detection step is shown in FIG. 8. A message “IN PROCESS OF DETECTION” is displayed in the information display region; at the same time, the STEP 3 tab automatically becomes active in place of the STEP 4 tab.

The STEP 3 tab is deactivated. Instead, the STEP 4 tab is activated. Since this step is final, the estimated necessary time for the step is consistent with the estimated necessary time for all steps.

When a new job is not added in the previous step (screen), the “ADDITION OF NEW JOB” button 212 is still active.

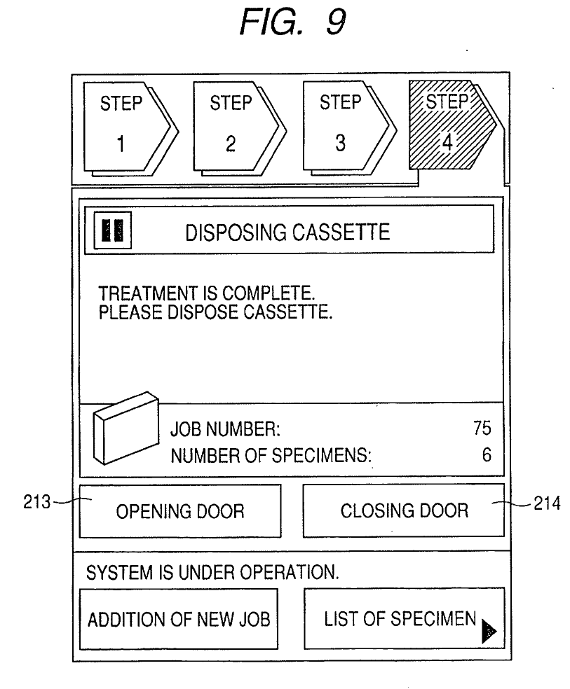

FIG. 9 shows a display screen at the time of finishing the testing item. The countdown for the estimated necessary time is not displayed in the information display region. The message “discard a cassette” is displayed and an “OPENING DOOR” button 213 and a “CLOSING DOOR” button 214 for a discarding port are displayed.

The user presses the “OPENING DOOR” button 213 to open the discarding door to take out the cassette and then presses the “CLOSING DOOR” button 214 to terminate the treatment.

If the cassette still remains when the door is closed, the same display screen is maintained and displays message “discard of the cassette” to urge the user to take out the cassette. When the cassette is discarded, the display screen returns to the initial screen (the initial state).

Now, the case where a new job is added while another job is under operation will be explained with reference to display screens.

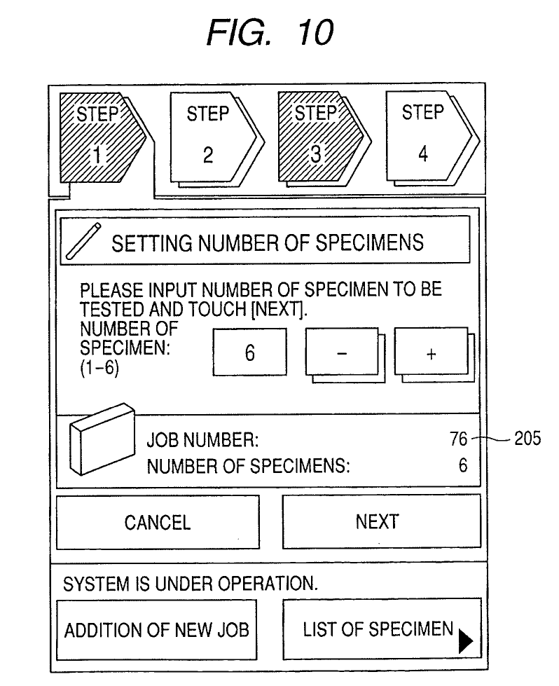

When the “ADDITION OF NEW JOB” button 212 (shown in FIG. 7) is pressed during the hybridization step, the display screen shown in FIG. 10 appears.

Since hybridization is in progress, the STEP 3 is under execution but deactivated. On the other hand, STEP 1 for the new job to be set is activated.

In the information display region of the STEP 1, the user sets the number of specimens to be set in this job in the same manner as in FIG. 3. At this time, the column of “job number” 205 (arranged in the lower portion of the information display region) is incremented from 75 to 76.

Thereafter, the step is initiated in the same procedure as described above. This time, a specimen(s) and the amplification reagent can be set in the apparatus; however, the hybridization reagent and the cassette having a DNA chip installed therein cannot be set since they are now in use.

Then, in the information display region, the estimated necessary time for the hybridization step of the previous job may be displayed as the reagent injecting time, although it is not shown in the figure.

By displaying the injecting time of the reagent and the setting time of DNA chip, the user understands which time the next operation is performed.

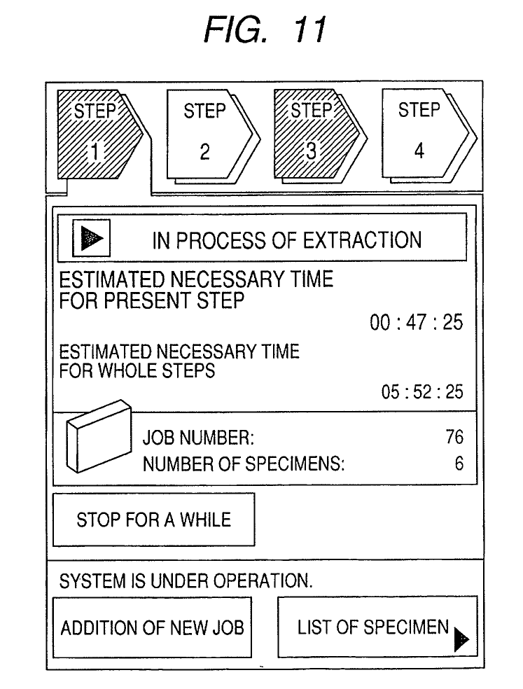

When the extraction step is started, the display screen shown in FIG. 11 appears. After the display screen is displayed for several seconds (e.g., 5 seconds), the display screen of the STEP 3 of the previous job automatically appears.

In this way, the information of the previous job is preferentially displayed. This is because it is important for the user to know the results of the screening of the previous job at the earliest possible time.

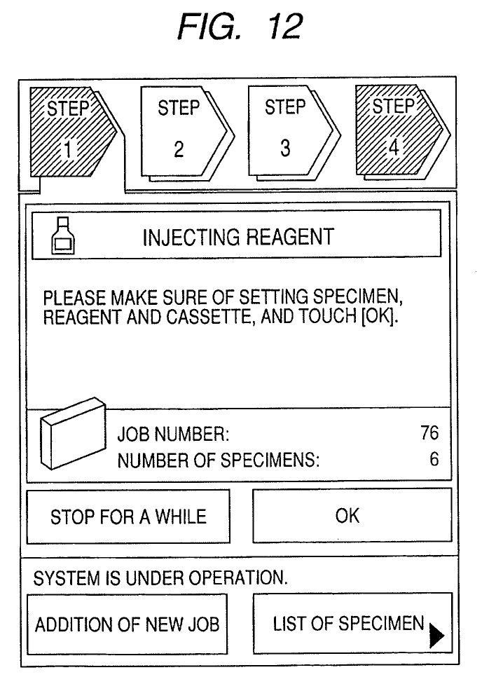

When the previous job proceeds to the detection step, a hybridization reagent and the cassette having a DNA chip installed therein (for the following job) can come to be set in the apparatus. At this time, the display screen shown in FIG. 12 appears. The message that the hybridization reagent can be injected is displayed on the screen, urging the user to inject the reagent.

To notify the user of the message, the STEP 1 tab for the job may be allowed to flash on and off or change to a warning color.

When a warning message and an instruction message are displayed, the corresponding index is also placed in a warning state. If so, the message that the information is not a simple message but a warning message can be clearly shown.

Since the previous job proceeds to the detection step, the STEP 4 tab shows that the step is in progress.

During the injecting operation of reagent and the setting operation of cassette, since the injecting and setting operations are nothing to do with the jobs in progress, the previous job proceeds in the detection step and the job presently on display is in process of the extraction step.

As described above, when the user inputs the message “a predetermined treatment is completed” while the message (of the previous job) is displayed, the index which corresponds to the step for the previous job switches to that of the following job, thereby displaying the information on the following job in the information display region.

Since the information of the previous job is always preferentially displayed at normal time as described above, the screening results next to be output from the apparatus can be obtained.

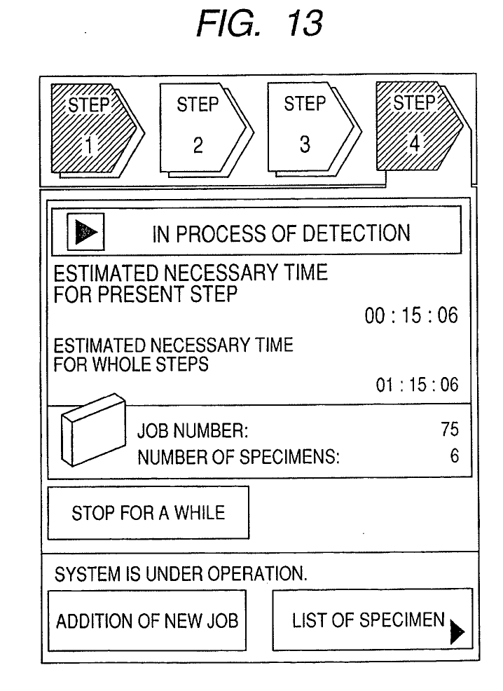

When the user sets the reagent and the cassette in the apparatus and presses the “OK” button, the display screen shown in FIG. 13 appears. As described above, the display automatically switches to the display of the previous job. At this time, when the user presses the STEP 1 tab, the information of the job in process of the extraction step can be displayed.

As described above, when the user presses a button showing the completion of operation, the treatment is completed. The treatment is completed not by the function of a sensor but by pressing the button. Even if the interruption of operation takes place, the operation can be clearly completed.

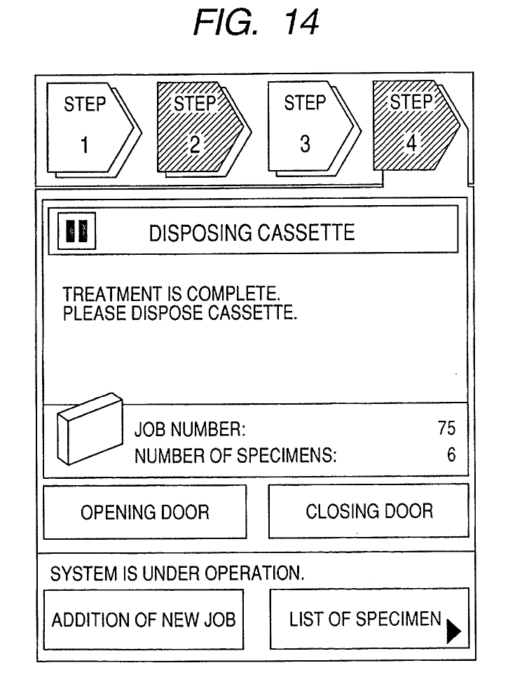

When the detection step of the previous job is completed, the screen shown in FIG. 14 appears. A message “discard the cassette” is displayed to urge the user to remove the cassette. The following job proceeds to the amplification step. The STEP 2 tab indicates the state of execution. This treatment (amplification) for the following job is continued regardless of the removing operation of the cassette.

However, when the following job proceeds and the hybridization step of the following job is completed, the following job cannot proceed to the detection step unless the user removes the cassette. In this case, treatment operation is interrupted until the cassette is removed.

To attract attention of the user, the STEP 4 tab for the following job may flash on and off or turn to a warning color so as to give warning to the user.

When the cassette is discarded, there are no tabs indicating that the previous job in progress. The tabs indicating the following job are continuously displayed.

By virtue of the constitution mentioned above, the degree of progress can be presented for on the job in progress. Even if a plurality of jobs are executed at the same time, their progress can be presented visually.

### EXAMPLE 2

Another embodiment will be explained with reference to the accompanying drawings.

As indexes used for displaying information on the display device 1604, tabs are used in Example 1 in the present invention.

However, the indexes of the present invention are not limited to the tabs. Any type of index may be used as long as it represents a processing step and provides the same effect.

A biochemical processing apparatus is used for treating DNA in Example 1; however, the apparatus may be used for other treatments. The display form must be varied depending upon the type of apparatus and degree of complication of the step. The type of index may be selected depending upon the type and size of display device 1604.

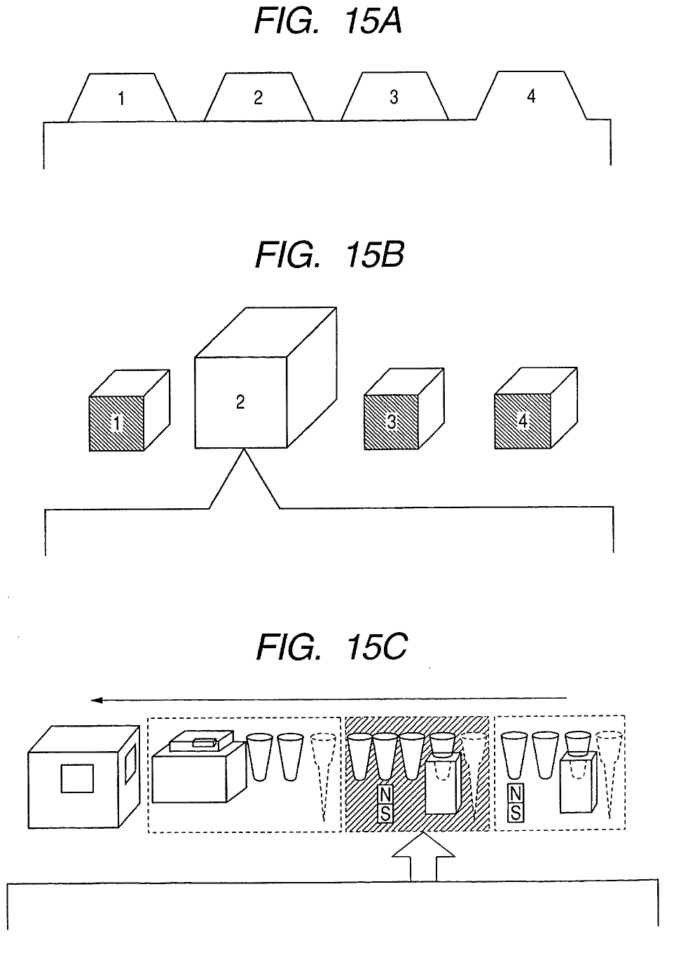

Types of indexes are shown in FIGS. 15A, 15B and 15C.

FIG. 15A shows general tab-form indexes. Modified tab indexes are used in Example 1. More specifically, tabs having caption letters thereon are arranged virtually or transversely. The information display region displays information in conjunction with the activated tab showing a step in progress.

A deactivated tab showing the step in progress can be indicated by changing the color to a lighter color than that of the activated tab. This is effective in the case where a display device is small.

FIG. 15B shows icon-form indexes. As shown in the example, box-form icons may be used. Alternatively, use may be made of icons using representative colors of the steps or icons using figures identifying the steps.

The color and size of an icon may be changed to indicate an unexecuted step. When an icon clearly identifies the step or when the user needs not to know detailed information of a step, caption letters may not be necessarily used.

It is preferable that the information display region points at the corresponding icon in the display screen. This is because the user easily to know that information displayed in the information display region is related to which step. This constitution is effective in the case where the processing steps of the apparatus can be easily iconified and the user can easily understand the content of the step by the icons.

The entire structural arrangement of the apparatus may be shown as indexes as shown in FIG. 15C. Since the structural arrangement of the apparatus are shown as they are, in which area of the apparatus the treatment is performed can be easily understood.

In the aforementioned two examples of indexes, when the indexes are arranged transversely, the operation of the steps proceeds the left to the right of the indexes. When the indexes are arranged vertically, the operation of the steps proceeds from the top to the bottom of the indexes.

However, it is not necessary that the operation of the apparatus actually proceeds as the order of the indexes. Therefore, the index showing the structure of the apparatus is effective for the user to have an image of the operation.

The order of the steps can be indicated by an arrow as shown in the figures. It is preferable that the information display region points at the step to which the information display region is concerned, as described above. This is because the step and the information display region can be linked with each other. This constitution is effective in the case where the steps and the structure of the apparatus are complicated.

As described in the foregoing, the display method of the present invention can be effective if the type of index is varied depending upon the characteristics of the apparatus and the size of the display device 1604.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2005-291228, filed Oct. 4, 2005, which is hereby incorporated by reference herein in its entirety.

What is claimed is:

1. A biochemical processing apparatus executing a plurality of processing steps, comprising
a display device; and
a display circuit for displaying an index corresponding to each of the processing steps and information regarding the processing step separately in different regions of the display device,
wherein the display circuit changes the display state of the index depending upon the progress of the processing step(s) and displays the information regarding the processing step corresponding to the index selected on the display device.

2. The biochemical processing apparatus according to claim 1, wherein the information regarding the processing step includes a warning and a message to the user.

3. The biochemical processing apparatus according to claim 2, wherein the display circuit shows information of a step of a previous job in the information display region when a plurality of steps are executed simultaneously.

4. The biochemical processing apparatus according to claim 2, wherein, when a warning and a massage are displayed, the user's attention is drawn by displaying the corresponding index thereof so as to give a warning.

5. The biochemical processing apparatus according to claim 1, wherein the index is a tab.

6. The biochemical processing apparatus according to claim 1, wherein the display device possesses a touch panel function.

7. The biochemical processing apparatus according to claim 6, wherein the index is selected by pressing the touch panel.

8. The biochemical processing apparatus according to claim 1, wherein the index is selected by operation of a mouse.

9. The biochemical processing apparatus according to claim 1, wherein the index is selected by operation of a specialized controller.

10. The biochemical processing apparatus according to claim 1, wherein the index is selected by pressing push buttons arranged around the display device.

11. The biochemical processing apparatus according to claim 1, wherein the information regarding the processing step includes the estimated time necessary for step in progress.

12. The biochemical processing apparatus according to claim 1, wherein the information regarding the processing step includes the estimated time necessary for the whole steps.

13. The biochemical processing apparatus according to claim 1, wherein the information regarding the processing step includes the time at which a reagent is injected and a DNA chip are set.

14. The biochemical processing apparatus according to claim 1, wherein the information regarding the processing step includes the number of specimens to be treated.

15. The biochemical processing apparatus according to claim 14, wherein the information regarding the processing step includes the identification numbers of specimen treatment.

16. The biochemical processing apparatus according to claim 1, wherein a step presently unexecuted, a selected step in progress, and a step in progress not selected are indicated by the index.

17. The biochemical processing apparatus according to claim 1, wherein the display circuit displays a system message of the entire apparatus in the region different from a region of the indexes and the information display region for displaying information regarding the processing step.
